(19) United States
(12) Patent Application Publication
(10) Pub. No.: US 2021/0052866 A1
(43) Pub. Date: Feb. 25, 2021

(54) MULTIPLEX CEREBROSPINAL FLUID PROCESSING SYSTEM

(71) Applicant: Aethlon Medical, Inc., San Diego, CA (US)

(72) Inventors: Craig P. Roberts, San Diego, CA (US); Michael Jacobs, San Diego, CA (US); James Joyce, San Diego, CA (US)

(21) Appl. No.: 16/636,297

(22) PCT Filed: Jul. 31, 2018
(86) PCT No.: PCT/US18/44576

§ 371 Date: Feb. 3, 2020

Related U.S. Application Data
(60) Provisional application No. 62/541,538, filed on Aug. 4, 2017.

(51) Int. Cl. A61M 27/00

(52) U.S. Cl. CPC A61M 27/006; A61M 2210/0693; A61M 2205/04; A61M 2205/36; A61M 2205/7509; A61M 2202/0028; A61M 2202/0208; A61M 2202/07; A61M 2202/206; A61M 2202/203; A61M 2205/7518

(57) ABSTRACT

Disclosed herein are cerebrospinal fluid (CSF) circulation systems and devices. Also disclosed are methods of using the systems and devices for the processing of CSF and for the treatment of brain or central nervous system diseases, disorders, and injuries. In particular, CSF may be processed to remove various molecules targets, including viruses, bacteria, exosomes, cells, proteins, debris, toxins, and inflammatory mediators. CSF may also be warmed, cooled, and/or oxygenated, and medications or substances may be added to the CSF for targeted treatment of a disease or disorder.

### FIELD OF THE INVENTION

The present disclosure relates generally to the field of medical devices. In particular, the disclosure relates to cerebrospinal fluid (CSF) circulation devices and systems and methods for processing CSF in a CSF circulation system. The devices, systems, and methods are useful for removal of CSF from a subject, circulation of the CSF outside of the body, treatment of the removed CSF, and returning the CSF to the body in real time.

### BACKGROUND

Cerebrospinal fluid (CSF) is a colorless body fluid found in the brain and spine. CSF is produced primarily in the choroid plexus of the brain, and serves to protect the brain's cortex by acting as a cushion and buffer by providing mechanical and immunological protection to the central nervous system. CSF protects the brain from injury during instances of impact and helps regulate intracranial pressure (ICP). CSF also acts as a transport medium for nutrients and provides chemical stability by removing metabolic waste from the central nervous system.

The human body contains from 100 to 160 mL of CSF at any given time. CSF comprises mainly water at a pH of 7.33, and contains various ions, such as sodium, potassium, calcium, magnesium, chloride, and glucose. CSF maintains an ICP in the physiologically normal range of 7 to 15 mmHg.

Various pathological conditions result in increased particles in the CSF. Furthermore, these conditions can result in increased ICP. Together, increased particles or debris in the CSF and increased ICP can result in decreased brain functioning. Current methods of screening or testing CSF are limited by the requirement of a lumbar puncture, which is negatively regarded by patients (Menendez-Gonzalez M. Routine lumbar puncture for the early diagnosis of Alzheimer's disease. Is it safe? Frontiers in Aging Neuroscience. 2014, 6). Furthermore, there is a lack of reliable methods for treating contaminated CSF so as to reduce the effects of brain disorders, diseases, or injuries. A system and method for processing large volumes of CSF from a subject is needed.

### SUMMARY

Provided herein are systems and devices for removing and circulating large volumes of cerebrospinal fluid (CSF). Also provided are methods for processing CSF, and methods for treating or inhibiting a brain disease, disorder, or injury.

Accordingly, provided herein is a CSF circulation device that comprises one or more catheters. In some embodiments, the CSF circulation device comprises a dual lumen catheter. In some embodiments, the dual lumen catheter comprises separate channels for CSF aspiration and for CSF return. In some embodiments the CSF circulation device comprises a first catheter that is configured to remove cerebrospinal fluid from a subject. In some embodiments, the first catheter is in a flow path with a second catheter configured to return processed cerebrospinal fluid to the subject. In some embodiments, the first catheter is configured to drain CSF from a subdural space, subarachnoid space, or brain ventricle. In some embodiments, the second catheter is configured to infuse CSF into a subdural space, subarachnoid space, or brain ventricle. In some embodiments, the one or more catheters include one or more pressure transducers configured to monitor and regulate intracranial pressure (ICP). In some embodiments the ICP is maintained at a range between 1 and 15 mmHg, such as 1, 2, 3, 4, 5, 6, 7, 8, 9, 10, 11, 12, 13, 14, or 15 mmHg or within a range defined by any two of the aforementioned pressures.

In some embodiments, the CSF circulation device includes at least one circulation tube in fluid communication with the one or more catheters.

In some embodiments, the CSF circulation device includes at least one column, membrane, and/or filter in fluid communication with the flow path. In some embodiments, the column, membrane, and/or filter can be in a unit separate from the device but associated therewith. In some embodiments, the at least one column, membrane, and/or filter is an affinity column, a capture column, a size exclusion column, or a column that includes one or more binding agents. In some embodiments, the at least one column, membrane, and/or filter includes a support that includes at least one binding molecule or affinity agent specific for viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof. In some embodiments, the binding molecule or affinity agent includes a lectin. In some embodiments, the lectin is selected from the group consisting of GNA, NPA, Concanavalin A, phytohemagglutinin, griffithsin, and cyanovirin. In some embodiments, the at least one column, membrane, and/or filter includes an antibody, a binding fragment of an antibody, an extracellular matrix protein, such as hyaluronic acid, laminin, or fibronectin, a sorbent, an activated carbon, a ligand, an aptamer, or an immobilized biochemical compound or any combination thereof. In some embodiments, the at least one column, membrane, and/or filter is configured to remove viruses, bacteria, parasites, fungi, exosomes, cells, proteins, toxins, inflammatory mediators, or fragments of viruses, bacteria, parasites, fungi, exosomes, cells, proteins, toxins, inflammatory mediators or a combination thereof.

In some embodiments, the one or more columns, membranes, and/or filters are configured to remove or capture a protein. In some embodiments, the protein is selected from the group consisting of prions, tau, amyloid beta, alpha synuclein, leucine-rich repeat kinase 2, and superoxide dismutase enzymes.

In some embodiments, the CSF circulation device includes at least one pump configured to circulate the cerebrospinal fluid through the flow path. In some embodiments, the at least one pump is configured to circulate the fluid at a flow rate that is within a range between 1 to 50 mL/min, such as 1, 2, 3, 4, 5, 6, 7, 8, 9, 10, 15, 20, 25, 30, 35, 40, 45, or 50 mL/min, or within a range defined by any two of the aforementioned flow rates. In some embodiments, the at least one pump includes a peristaltic pump, a diaphragm pump, or a piston pump.

In some embodiments, the CSF circulation device comprises one or more filters. In some embodiments, the one or more filters comprise one or more capsule filters, disc filters, or combinations thereof. In some embodiments, the one or more filters are provided in tandem e.g., as a single filtration unit. In some embodiments, the one or more filters comprises a dual capsule filter. In some embodiments, the one or more filters comprises a size exclusion filter, an adsorption filter, an affinity filter, a capture filter, or a combination thereof. In some embodiments, the one or more filters is configured for filtration of particles, large molecules, small molecules, target molecules, such as prions, tau, amyloid beta, alpha synuclein, leucine-rich repeat kinase 2, and/or superoxide dismutase enzymes or any combinations thereof. In some embodiments, the one or more filters comprise a polymer, such as polyether sulfone. In some embodiments, the one or more filters comprise a pore size that is in the range of 0.05, 0.1, 0.2, 0.3, 0.4, 0.5, 0.6, 0.7, 0.8, 0.9, or 1.0 μm, or within a range defined by any two of the aforementioned pore sizes.

In some embodiments, the CSF circulation device includes a heat exchanger configured to control the temperature of the fluid in the flow path. In some embodiments, the heat exchanger maintains the CSF at a temperature in a range from 10 to 42° C., such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42° C. or within a range defined by any two of the aforementioned temperatures.

In some embodiments, the CSF circulation device comprises an oxygenator. In some embodiments, CSF is oxygenated with an oxygen range from 21-100% oxygen, such as 21, 22, 23, 24, 25, 30, 35, 40, 45, 50, 55, 60, 65, 70, 75, 80, 85, 90, 95, or 100% or within a range defined by any two of the aforementioned percentages.

In some embodiments, the CSF circulation device comprises a port that is configured to receive an additive and/or remove cerebrospinal fluid or a component thereof. In some embodiments, the additive is a fluid, a medicament, or a drug, such as a chemotherapeutic, a steroid, an antibiotic, or an antibody, or a drug delivery vehicle comprising a drug for treating the brain disorder, disease, or injury. In some embodiments, the additive comprises exosomes, which comprise a medicament, a nucleic acid, or drug.

In some embodiments, a method for processing CSF in a CSF circulation device is provided. In some embodiments, the method comprises introducing CSF from a subject into a CSF processing device or system as described herein. In some embodiments, the method comprises passing the CSF through the at least one column, membrane, and/or filter of the CSF processing device. In some embodiments, the column, membrane, and/or filter are in a unit separate from the device but the CSF is passed through the column, membrane, or filter in the method. In some embodiments, the CSF is returned to the subject after passage through the at least one column, membrane, and/or filter or passage through the CSF processing device itself.

In some embodiments, the CSF is removed from a subdural space, a subarachnoid space, or a brain ventricle of the subject by one or more catheters. In some embodiments, the CSF that passes through the at least one column, membrane, and/or filter and/or CSF processing device itself has fewer particles, e.g., viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof than before introduction of the CSF into the device.

In some embodiments, a method for circulating cerebrospinal fluid through a CSF circulation device or system is provided wherein, the method comprises contacting a patient or subject with the CSF circulation device and circulating the CSF of a subject through said circulation device. In some embodiments, the CSF that passes through the circulation device also passes through a column, membrane, and/or filter and, in some embodiments, the CSF from the patient or subject is returned to the patient or subject after passage through the device, optionally, after passage through the column, membrane, and/or filter. In some embodiments, the column, membrane, and/or filter are in a unit separate from the device but the CSF is passed through the column, membrane, or filter in the method.

In some embodiments, a method of removing viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof from the CSF of a subject or patient is provided. In some embodiments, the method comprises introducing CSF from a subject or patient into the CSF circulation device, as described herein. In some embodiments, the method comprises circulating the CSF that is introduced into the device, optionally through a column, membrane, or filter such that the CSF has fewer viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof than it had prior to introduction into the device. In some embodiments, the column, membrane, and/or filter are in a unit separate from the device but the CSF is passed through the column, membrane, or filter in the method. In some embodiments, the CSF that passes through the circulation device has a reduced quantity of particles. In some embodiments, the particles comprise viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof. In some embodiments, the CSF is subjected to a temperature change. In some embodiments, the cerebrospinal fluid is oxygenated. In some embodiments, the cerebrospinal fluid is medicated or an additive is added to the CSF prior to introduction into the subject or patient, including a steroid, antibiotic, or antibody medication, or a drug delivery vehicle comprising a drug for treating the brain disorder, disease, or injury. In some embodiments, the additive comprises exosomes, which comprise a medicament, a nucleic acid, or drug.

In some embodiments, a method of treating or inhibiting a bacterial and/or viral infection of the cerebrospinal fluid or brain of a subject or patient, such as encephalitis or meningitis, is provided. In some embodiments, the method comprises introducing CSF from a subject or patient into the CSF circulation device or system, as described herein. In some embodiments, the method comprises circulating the CSF of the subject through said circulation device, and optionally the device further comprises a column, membrane, and/or filter, such that the CSF has fewer virus, or bacteria or fragments thereof than it had prior to introduction into the device. In some embodiments, the column, membrane, and/or filter are in a unit separate from the device but the CSF is passed through the column, membrane, or filter in the method. In some embodiments, the method comprises returning the CSF to the subject, preferably after the CSF has passed through a column, membrane, and/or filter present in the device. In some embodiments, the CSF has fewer inflammatory molecules than it had prior to introduction into the device. In some embodiments, the CSF is cooled to a temperature of 20° C. In some embodiments, the cerebrospinal fluid is medicated or an additive is added to the CSF prior to introduction into the subject or patient, including a steroid, antibiotic, or antibody medication, or a drug delivery vehicle comprising a drug for treating the bacterial and/or viral infection. In some embodiments, the additive comprises exosomes, which comprise a medicament, a nucleic acid, or drug.

In some embodiments a method of treating, reducing, or ameliorating a brain injury such as a traumatic brain injury, in a subject is provided. In some embodiments, the method comprises introducing CSF from a subject into the CSF circulation device or system as described herein. In some embodiments, the method comprises circulating the CSF of the subject through said circulation device such that the CSF has fewer exosomes and/or proteins associated with traumatic brain injury than it had prior to introduction into the device. Optionally, the method is practiced by circulating the CSF through the circulation device that also comprises a column, membrane, or filter. In some embodiments, the column, membrane, and/or filter are in a unit separate from the device but the CSF is passed through the column, membrane, or filter in the method. In some embodiments, the method comprises returning the CSF to the subject, preferably after circulation through a column, membrane, and/or filter. In some embodiments, the CSF has less cellular debris, proteins, or fragments thereof than it had prior to introduction into the device. In some embodiments, the exosomes or proteins associated with traumatic brain injury comprise tau, amyloid beta, prions, or fragments or combinations thereof. In some embodiments, the CSF is cooled to a temperature of 20° C. In some embodiments, the cerebrospinal fluid is medicated or an additive is added to the CSF prior to introduction into the subject or patient, including a steroid, antibiotic, or antibody medication, or a drug delivery vehicle comprising a drug for treating the brain injury. In some embodiments, the additive comprises exosomes, which comprise a medicament, a nucleic acid, or drug.

In some embodiments a method of treating, reducing, or inhibiting one or more abscesses in a subject is provided. In some embodiments, the method comprises introducing CSF from a subject into the CSF circulation device or system as described herein. In some embodiments, the method comprises circulating the CSF of the subject through said circulation device such that the CSF has fewer free floating cells associated with the one or more abscesses than it had prior to introduction into the device. Optionally, the method is practiced by circulating the CSF through the circulation device that also comprises a column, membrane, or filter. In some embodiments, the column, membrane, and/or filter are in a unit separate from the device but the CSF is passed through the column, membrane, or filter in the method. In some embodiments, the method includes returning the CSF to the subject, preferably after passage through the column, membrane, and/or filter. In some embodiments, the method includes providing, adding or administering a medication or additive to the CSF during circulation through the device so as to promote the reduction, inhibition, or treatment of the one or more abscesses. In some embodiments, the medication or additive, such as a steroid, antibody, or other medicament, or a drug delivery vehicle comprising a drug for treating the brain abscess, is added to the CSF e.g., through a port on the device, during or after circulation and prior to returning the CSF to the subject. In some embodiments, the medication includes antibiotics or a drug delivery vehicle comprising an antibiotic. In some embodiments, the additive comprises exosomes, which comprise a medicament, a nucleic acid, or drug. In some embodiments, the cerebrospinal fluid is cooled to a temperature of 20° C.

In some embodiments, a method of treating, inhibiting, or ameliorating a degenerative neurological disease in a subject or patient is provided. In some embodiments, the method comprises introducing CSF from a subject into the CSF circulation device or system, as described herein. In some embodiments, the CSF of the subject is circulated through said circulation device such that the CSF has fewer particles associated with the degenerative neurological disease than it had prior to introduction into the device. Optionally, the method is practiced by circulating the CSF through the circulation device that also comprises a column, membrane, or filter. In some embodiments, the column, membrane, and/or filter are in a unit separate from the device but the CSF is passed through the column, membrane, or filter in the method. In some embodiments, the CSF is returned to the subject, preferably after passage through the column, membrane, or filter. In some embodiments, the degenerative neurological disease is selected from the group consisting of hemorrhagic stroke, chronic traumatic encephalopathy, Alzheimer's disease, Parkinson's disease, Huntington's disease, and amyotrophic lateral sclerosis. In some embodiments, the cerebrospinal fluid is medicated or an additive is added to the CSF prior to introduction into the subject or patient, including a steroid, antibiotic, or antibody medication, or a drug delivery vehicle comprising a drug for treating the brain disorder, disease, or injury. In some embodiments, the additive comprises exosomes, which comprise a medicament, a nucleic acid, or drug.

In some embodiments, a method of treating, inhibiting, or ameliorating a target pathology in a subject or patient is provided. In some embodiments, the target pathology is selected from the group consisting of fungi, parasites, protozoa, and prions. In some embodiments, the method includes introducing CSF from a subject into the CSF circulation device or system, as described herein. In some embodiments, the CSF of the subject is circulated through said circulation device such that the CSF has less of the target pathology than it had prior to introduction into the device. Optionally, the method is practiced by circulating the CSF through the circulation device that also comprises a column, membrane, or filter. In some embodiments, the column, membrane, and/or filter are in a unit separate from the device but the CSF is passed through the column, membrane, or filter in the method. In some embodiments, the CSF is returned to the subject, preferably after the CSF has passed through the column, membrane, or filter. In some embodiments, the method includes providing, adding, or administrating a medication or additive to the CSF during or after circulation through the device e.g., through a port on the device. In some embodiments, the medication or additive, such as a steroid, antibody, or antibiotic, or a drug delivery vehicle comprising a drug for treating the brain disorder, disease, or injury is added to the CSF after circulation and prior to returning the CSF to the subject. In some embodiments, the additive comprises exosomes, which comprise a medicament, a nucleic acid, or drug. In some embodiments, the cerebrospinal fluid is cooled to a temperature of 20° C.

In some embodiments a method of removing viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof from CSF of a subject or patient is provided. In some embodiments, the method includes introducing CSF from a subject into the CSF circulation device or system, as described herein. In some embodiments, the CSF of the subject is circulated through said circulation device such that the CSF has less of the viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof than it had prior to introduction into the device. Optionally, the method is practiced by circulating the CSF through the circulation device that also comprises a column, membrane, or filter. In some embodiments, the column, membrane, and/or filter are in a unit separate from the device but the CSF is passed through the column, membrane, or filter in the method. In some embodiments, the column, membrane, or filter comprises a support that comprises an affinity binding molecule. In some embodiments, the CSF is recovered after contact with the column, membrane, or filter. In some embodiments, the affinity binding molecule includes a lectin selected from the group consisting of GNA, NPA, Concanavalin A, phytohemagglutinin, griffithsin, and cyanovirin or an antibody or binding fragment thereof or an extracellular matrix protein. In some embodiments, the CSF is returned to the subject, preferably after the CSF has passed through the column, membrane, or filter. In some embodiments, the method includes providing, adding, or administrating a medication or additive to the CSF during or after circulation through the device e.g., through a port on the device. In some embodiments, the medication or additive, such as a steroid, antibody, or antibiotic, or a drug delivery vehicle comprising a drug for treating the brain disorder, disease, or injury, is added to the CSF after circulation and prior to returning the CSF to the subject. In some embodiments, the additive comprises exosomes, which comprise a medicament, a nucleic acid, or drug. In some embodiments, the cerebrospinal fluid is cooled to a temperature of 20° C.

In some embodiments, a method of treating, inhibiting, or ameliorating a brain cancer or brain tumor in a subject or patient is provided. In some embodiments, the method includes introducing CSF from a subject into the CSF circulation device or system, as described herein. In some embodiments, the CSF of the subject is circulated through said circulation device such that the CSF has less cellular debris (e.g., fewer cell membranes after circulation through said circulation device), toxins, inflammatory mediators, cytokines, or other exogenous particulate than it had prior to introduction into the device. Optionally, the method is practiced by circulating the CSF through the circulation device that also comprises a column, membrane, or filter. In some embodiments, the column, membrane, and/or filter are in a unit separate from the device but the CSF is passed through the column, membrane, or filter in the method. In some embodiments, the CSF is returned to the subject, preferably after the CSF has passed through the column, membrane, or filter. In some embodiments, the method includes providing, adding, or administrating a medication or additive, such as a steroid, chemotherapeutic, antibody, or other medicament, or a drug delivery vehicle comprising a drug for treating the brain cancer or brain tumor, to the CSF during or after circulation through the device e.g., through a port on the device. In some embodiments, the medication or additive, such as a steroid, chemotherapeutic, antibody, or other medicament, or drug delivery vehicle comprising a drug, is added to the CSF after circulation and prior to returning the CSF to the subject. In some embodiments, the additive comprises exosomes, which comprise a medicament, a nucleic acid, or drug. In some embodiments, the cerebrospinal fluid is cooled to a temperature of 20° C.

Accordingly, some aspects described herein relate to the following alternatives:

1. A cerebrospinal fluid circulation device comprising:
one or more catheters;
at least one circulation tube in fluid communication with the one or more catheters;
at least one column in fluid communication with the flow path; and
at least one pump configured to circulate the cerebrospinal fluid through the flow path.

2. The device of alternative 1, wherein the one or more catheters comprise a first catheter configured to remove cerebrospinal fluid from a subject, in a flow path with a second catheter configured to return processed cerebrospinal fluid to the subject, wherein the first catheter is configured for drainage from a subarachnoid space or ventricle, and wherein the second catheter is configured for infusion into a subarachnoid space or ventricle.

3. The device of alternative 1, wherein the one or more catheters comprise a dual lumen catheter.

4. The device of alternative 1, wherein the at least one column is an affinity column, a capture column, or a column that comprises one or more binding agents.

5. The device of alternative 4, wherein the at least one column comprises a support that comprises at least one binding molecule or affinity agent specific for viruses, bacteria, parasites, fungi, exosomes, cells, proteins, toxins, inflammatory mediators, or fragments of viruses, bacteria, parasites, fungi, exosomes, cells, proteins, toxins, or inflammatory mediators or any combination thereof.

6. The device of alternative 6, wherein the binding molecule or affinity agent comprises a lectin.

7. The device of alternative 6, wherein the lectin is selected from the group consisting of GNA, NPA, Concanavalin A, phytohemagglutinin, griffithsin, and cyanovirin.

8. The device of any one of alternatives 1-7, wherein the at least one column comprises an antibody, a binding fragment of an antibody, an extracellular matrix protein, such as hyaluronic acid, laminin, or fibronectin, a sorbent, an activated carbon, a ligand, an aptamer, or an immobilized biochemical compound or any combination thereof.

9. The device of any one of alternatives 1-8, wherein the at least one column is configured to remove viruses, bacteria, parasites, fungi, exosomes, cells, proteins, toxins, inflammatory mediators, or fragments of viruses, bacteria, parasites, fungi, exosomes, cells, proteins, toxins, inflammatory mediators or a combination thereof.

10. The device of alternative 9, wherein the protein is selected from the group consisting of prions, tau, amyloid beta, alpha synuclein, leucine-rich repeat kinase 2, and superoxide dismutase enzymes.

11. The device of any one of alternatives 1-10, wherein the one or more catheters comprise one or more pressure transducers configured to monitor and regulate intracranial pressure.

12. The device of alternative 11, wherein the intracranial pressure is maintained at a range between 1 and 15 mmHg, such as 1, 2, 3, 4, 5, 6, 7, 8, 9, 10, 11, 12, 13, 14, or 15 mmHg or within a range defined by any two of the aforementioned pressures.

13. The device of any one of alternatives 1-12, wherein the at least one pump is configured to circulate the fluid at a flow rate that is within a range between 1 to 50 mL/min, such as 1, 2, 3, 4, 5, 6, 7, 8, 9, 10, 15, 20, 25, 30, 35, 40, 45, or 50 mL/min, or within a range defined by any two of the aforementioned flow rates.

14. The device of any one of alternatives 1-13, wherein the at least one pump comprises a peristaltic pump, a diaphragm pump, or a piston pump.

15. The device of any one of alternatives 1-14, further comprising one or more filters.

16. The device of alternative 15, wherein the one or more filters comprise one or more capsule filters or disc filters.

17. The device of any one of alternatives 15-16, wherein the one or more filters are provided in tandem e.g., as a single filtration unit.

18. The device of any one of alternatives 15-17, wherein the one or more filters comprises a dual capsule filter.

19. The device of any one of alternatives 15-18, wherein the one or more filters comprises a size exclusion filter, an adsorption filter, or an affinity filter, a capture filter, or a combination thereof.

20. The device of any one of alternatives 15-19, wherein the one or more filters is configured for filtration of particles, large molecules, small molecules, target molecules, such as prions, tau, amyloid beta, alpha synuclein, leucine-rich repeat kinase 2, and/or superoxide dismutase enzymes or any combinations thereof.

21. The device of any one of alternatives 15-20, wherein the one or more filters comprise a polymer, such as polyether sulfone.

22. The device of any one of alternatives 15-21, wherein the one or more filters comprise a pore size in the range of 0.05, 0.1, 0.2, 0.3, 0.4, 0.5, 0.6, 0.7, 0.8, 0.9, or 1.0 μm, or within a range defined by any two of the aforementioned pore sizes.

23. The device of any one of alternatives 1-22, further comprising a heat exchanger configured to control the temperature of the fluid in the flow path.

24. The device of alternative 23, wherein the cerebrospinal fluid is maintained at a temperature in a range from 10 to 42° C., such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42° C. or within a range defined by any two of the aforementioned temperatures.

25. The device of any one of alternatives 1-24, further comprising an oxygenator.

26. The device of alternative 25, wherein the cerebrospinal fluid is oxygenated with an oxygen range from 21-100% oxygen, such as 21, 22, 23, 24, 25, 30, 35, 40, 45, 50, 55, 60, 65, 70, 75, 80, 85, 90, 95, or 100% or within a range defined by any two of the aforementioned percentages.

27. The device of any one of alternatives 1-26, further comprising a port configured to receive an additive and/or remove cerebrospinal fluid or a component thereof.

28. The device of alternative 27, wherein the additive is a drug or a fluid.

29. A method for processing cerebrospinal fluid in a cerebrospinal fluid circulation device of any one of alternatives 1-28 comprising:
(a) introducing cerebrospinal fluid from a subject into a device of anyone of alternatives 1-24;
(b) passing the cerebrospinal fluid through the at least one column of said device; and
(c) returning the cerebrospinal fluid to the subject after step (b).

30. The method of alternative 29, wherein the cerebrospinal fluid is removed from a subdural space, subarachnoid space, or ventricle of the subject by one or more catheters.

31. The method of alternative 30, wherein the one or more catheters comprise one or more pressure transducers configured to monitor intracranial pressure.

32. The method of any one of alternatives 29-31, wherein the intracranial pressure is maintained at a pressure of between 1 to 15 mmHg, such as 1, 2, 3, 4, 5, 6, 7, 8, 9, 10, 11, 12, 13, 14, or 15 mmHg or within a range defined by any two of the aforementioned pressures.

33. The method of any one of alternatives 29-32, wherein the cerebrospinal fluid is circulated at a flow rate in the range from 1 to 50 mL/min, such as 1, 2, 3, 4, 5, 6, 7, 8, 9, 10, 15, 20, 25, 30, 35, 40, 45, or 50 mL/min, or within a range defined by any two of the aforementioned flow rates.

34. The method of alternative 33, wherein the flow rate is 40 mL/min.

35. The method of any one of alternatives 29-34, wherein the processed cerebrospinal fluid has fewer exosomes, particles, virus, or bacteria than before introduction into the device.

36. A method for circulating cerebrospinal fluid comprising:
contacting a patient with the cerebrospinal fluid circulation device according to any one of alternatives 1-28;
circulating cerebrospinal fluid of a subject through said circulation device; and
returning the cerebrospinal fluid to the subject.

37. A method of removing exosomes, particles, virus, or bacteria from cerebrospinal fluid of a subject, comprising:
introducing cerebrospinal fluid from a subject into the device according to any one of alternatives 1-28; and
circulating the cerebrospinal fluid of the subject through said circulation device such that the cerebrospinal fluid has fewer exosomes, particles, virus, or bacteria than it had prior to introduction into the device.

38. The method of alternative 37, wherein the processed cerebrospinal fluid comprises a reduced quantity of particles.

39. The method of any one of alternative 37-38, wherein the particles comprise viruses, bacteria, exosomes, cells, proteins, debris, toxins, inflammatory mediators, or fragments or combinations thereof.

40. The method of any one of alternatives 37-39, wherein the cerebrospinal fluid is subjected to a temperature change.

41. The method of any one of alternatives 37-40, wherein the cerebrospinal fluid is oxygenated.

42. The method of any one of alternatives 37-41, wherein the cerebrospinal fluid is medicated.

43. A method of treating or inhibiting a bacterial and/or viral infection of the cerebrospinal fluid or brain, such as encephalitis or meningitis, in a subject comprising:
introducing cerebrospinal fluid from a subject into the device according to any one of alternatives 1-28;
circulating the cerebrospinal fluid of the subject through said circulation device such that the cerebrospinal fluid has fewer virus, or bacteria than it had prior to introduction into the device; and
returning the cerebrospinal fluid to the subject.

44. The method of alternative 43, wherein the cerebrospinal fluid has fewer inflammatory molecules than it had prior to introduction into the device.

45. The method of any one of alternatives 43-44, wherein the cerebrospinal fluid is cooled to a temperature of 20° C.

46. A method of treating or ameliorating a traumatic brain injury in a subject, comprising:
introducing cerebrospinal fluid from a subject into the device according to any one of alternatives 1-28;
circulating the cerebrospinal fluid of the subject through said circulation device such that the cerebrospinal fluid has fewer exosomes and/or proteins associated with traumatic brain injury than it had prior to introduction into the device; and
returning the cerebrospinal fluid to the subject.

47. The method of alternative 46, wherein the cerebrospinal fluid has less cellular debris, proteins, or fragments thereof than it had prior to introduction into the device.

48. The method of any one of alternatives 46-47, wherein the exosomes or proteins associated with traumatic brain injury comprise tau, amyloid beta, prions, or fragments or combinations thereof.

49. The method of any one of alternatives 46-48, wherein the processed cerebrospinal fluid is cooled to a temperature of 20° C.

50. A method of treating or inhibiting one or more abscesses in a subject, comprising:
introducing cerebrospinal fluid from a subject into the device according to any one of alternatives 1-28;
circulating the cerebrospinal fluid of the subject through said circulation device such that the cerebrospinal fluid has fewer free floating cells associated with the one or more abscesses than it had prior to introduction into the device; and
returning the cerebrospinal fluid to the subject.

51. The method of alternative 50, further comprising administration of medication for the treatment of the one or more abscesses.

52. The method of alternative 51, wherein the medication comprises antibiotics, antibodies, chemotherapeutics, steroids, exosomes, or drug delivery vehicles.

53. The method of any one of alternative 50-52, wherein the cerebrospinal fluid is cooled to a temperature of 20° C.

54. A method of treating or ameliorating a degenerative neurological disease in a subject, comprising:
introducing cerebrospinal fluid from a subject into the device according to any one of alternatives 1-28;
circulating the cerebrospinal fluid of the subject through said circulation device such that the cerebrospinal fluid has fewer particles associated with the degenerative neurological disease than it had prior to introduction into the device; and
returning the cerebrospinal fluid to the subject.

55. The method of alternative 54, wherein the degenerative neurological disease is selected from the group consisting of hemorrhagic stroke, chronic traumatic encephalopathy, Alzheimer's disease, Parkinson's disease, and amyotrophic lateral sclerosis.

56. A method of treating or ameliorating a target pathology in a subject, comprising:
introducing cerebrospinal fluid from a subject into the device according to any one of alternatives 1-28;
circulating the cerebrospinal fluid of the subject through said circulation device such that the cerebrospinal fluid has less of the target pathology than it had prior to introduction into the device; and
returning the cerebrospinal fluid to the subject.

57. The method of alternative 56, wherein the target pathology is selected from the group consisting of fungi, parasites, protozoa, and prions.

58. The method of any one of alternatives 56-57, further comprising administration of a medication.

59. The method of any one of alternatives 56-58, wherein the cerebrospinal fluid is cooled to a temperature of 20° C.

60. A method of removing exosomes, particles, virus, or bacteria from cerebrospinal fluid of a subject, comprising:
contacting cerebrospinal fluid from a subject with a support that comprises an affinity binding molecule; and
recovering the cerebrospinal fluid.

61. The method of alternative 60, further comprising returning the recovered cerebrospinal fluid to the subject.

62. The method of alternative 60, wherein the affinity binding molecule comprises a lectin selected from the group consisting of GNA, NPA, Concanavalin A, phytohemagglutinin, griffithsin, and cyanovirin.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments, and are not intended to be limiting in scope.

FIG. 1 illustrates a cerebrospinal fluid circulation system. The system is CSF circulation system comprising catheters and circulation tube. A pump circulates the CSF through the system, where the CSF is processed by passing through processing column. The CSF may be additionally treated by the addition of fluids or medicaments, the removal of fluid, or by varying the temperature and/or oxygenation of the CSF.

FIG. 2 illustrates a schematic diagram of an exemplary embodiment of a dual capsule filter system containing a binding agent, configured for capture of molecules of interest from CSF.

### DETAILED DESCRIPTION

Provided herein is a cerebrospinal fluid circulation system or device for processing, cleansing, and/or circulating CSF. In various disease conditions, CSF becomes contaminated or damaged with particulate matter, which can include, for example, viruses, bacteria, exosomes, cells, proteins, debris, toxins, inflammatory mediator, or fragments or combinations thereof. Contaminated or damaged CSF is a result of various brain and central nervous system (CNS) conditions, including diseases, disorders, or injuries of the brain. Contaminated or damaged CSF can further exacerbate the condition, and may cause a general decrease in brain and/or CNS function. Furthermore, particulate matter in the CSF produces inflammation, increased ICP, and decreased CSF oxygen levels, resulting in a loss of neuronal oxygenation, and decreased neuronal functioning. The cerebrospinal fluid circulation system provided herein allows for the processing, cleansing, and/or circulation of CSF, thereby facilitating the removal of particulate matter from CSF, drug delivery into the cranium beyond the blood brain barrier, reduction in ICP, cooling or warming of CSF, and oxygenation of CSF.

Traditional systems for screening or processing CSF employ a method of accessing CSF through a spinal access site, such as in the lumbar CSF space. However, a lumbar puncture is a procedure that remains controversial, and is a complicated process that is negatively regarded by patients. Furthermore, a common sequel to lumbar puncture is post-lumbar puncture headaches, with symptoms commonly lasting several days and often severe enough to immobilize the subject. Furthermore, such headaches can be fatal if left untreated as a result of complications such as subdural hematoma and seizures. Post-lumbar puncture headaches are generally a result of a change in the ICP caused by removal of CSF.

Accordingly, provided herein is a CSF processing system that gains access to CSF in the subdural space, subarachnoid space, or ventricle. The systems and methods provided herein overcome the shortcomings of traditional devices for screening or processing CSF. The CSF processing system provided herein provides increased efficiency of CSF processing, and improves the ability to process and cleanse CSF, by providing the capability to remove particulate matter from CSF, to administer drugs or other medications to the brain through the CSF, to reduce ICP, to cool or warm CSF, to oxygenate CS, or to otherwise treat and process CSF.

### Definitions

Unless otherwise defined, all technical and scientific terms used herein have the same meaning as commonly understood by one of ordinary skill in the art to which this present application belongs. Although methods and materials similar to those described herein can be used in the practice or testing of the present application, suitable methods and materials are described below. All publications, patent applications, patents, and other references mentioned herein are incorporated in the application by reference in their entirety. In addition, the materials, methods, and examples are illustrative only and not intended to be limiting. For purposes of the present disclosure, the following terms are defined below.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “treatment” refers to an intervention made in response to a disease, disorder or physiological condition manifested by a subject, particularly a subject suffering from a neurological disorder or a disorder affecting the nervous system. Such disorders can include, but are not limited to, neurodegenerative diseases (including, e.g., Alzheimer's disease, Parkinson's disease, chronic traumatic encephalopathy, amyotrophic lateral sclerosis), brain injuries, such as traumatic brain injuries, strokes, abscesses, infections of the central nervous system (including, e.g., virus, bacterium, fungus, parasite, protozoan, and prion infections), tumors, multiple sclerosis, and autoimmune diseases (including, e.g., Guillain-Barre syndrome) and other disorders affecting the nervous system, among others described herein and known in the art. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments, treatments reduce, alleviate, or eradicate the symptom(s) of the disease(s). As used herein, the term “prevention” refers to any activity that reduces the burden of the individual later expressing disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

As used herein, the term “subject” is an animal, such as a vertebrate, preferably a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the subject is mouse or rat. In some embodiments, the subject is a primate, such as a non-human primate or a human. A “subject” includes any animal that exhibits a symptom, or is at risk for exhibiting a symptom, of one or more disorder described herein. Suitable subjects (patients) include laboratory animals (such as mouse, rat, rabbit, or guinea pig), farm animals, and domestic animals or pets (such as a cat or dog). Non-human primates and, preferably, human patients, are included.

### Cerebrospinal Fluid

Cerebrospinal fluid is important for the regulation of brain functioning. CSF can become contaminated, which results in the improper functioning of CSF, and a decreased ability to protect the brain. Furthermore, the contaminants themselves contribute to disease progression. CSF is a colorless body fluid found in the brain and spine, and which circulates within the ventricular system of the brain. CSF offers protection to the central nervous system by acting as both a mechanical and chemical buffer. As a mechanical buffer, CSF provides a somewhat limited cushion to the brain by protecting the brain tissue from injury when jolted. As a chemical buffer, CSF clears metabolic waste from the central nervous system.

Diagnosis of several diseases or disorders of the brain can be made by examining CSF for contaminants or debris, including, for example, examination for the presence of viruses, bacteria, parasites, prions, fungi, proteins, glucose levels, or CSF pressure. In some embodiments, a patient or subject is selected or identified as one in need of the removal of one or more CSF contaminants and such a selection or identification is performed using clinical or diagnostic tools that identify the presence of such contaminants.

The brain is protected by a highly selected permeability barrier referred to as the blood brain barrier (BBB), which separates circulating blood from the brain extracellular fluid. The BBB protects the brain from most pathogens, but allows passive diffusion of selective molecules that are crucial for neural functioning. Many medications, including for example, antibodies and certain antibiotics, are unable to cross the BBB. However, drugs administered directly to CSF can enter the brain by crossing the blood-cerebrospinal fluid barrier. Accordingly, drugs administered through the CSF circulation system described herein are capable of bypassing the BBB, and therefore, higher concentrations of medication are able to reach the target area than through conventional administration routes, for example intravenous administration.

### Cerebrospinal Fluid Processing System

As used herein, the term “processing system” refers broadly to a system for processing bodily material of a subject. In some embodiments is a system for processing CSF. In some embodiments, the CSF is processed outside of the body. A CSF circulation system or a CSF processing system refers to a processing system or device that circulates or processes CSF. In some embodiments, the CSF circulation device may include various components, which may include, for example, one or more catheters, one or more pumps, one or more pressure sensors, one or more filters, one or more column, one or more oxygenator, one or more heat exchanger, one or more ports for access to the CSF, or combinations thereof. These various components are described in greater detail below.

Catheters

As used herein, the term “catheters” refers to a catheter for use in the CSF processing system. In some embodiments, the CSF processing system includes one or more catheters. In some embodiments, the CSF processing system includes a single catheter configured as a dual lumen catheter. In some embodiments, a dual lumen catheter comprises dual lumens, or channels, whereby fluid may be aspirated through a first lumen, and may be returned through a second lumen. Thus, in some embodiments, a dual lumen catheter is used for both aspiration and infusion of CSF. Use of a single catheter requires a single entry point to gain access to CSF.

In some embodiments, the CSF processing system includes a first and a second catheter. In some embodiments, the first catheter is a drainage catheter configured for removal of CSF from the subdural space, subarachnoid space, or ventricle. In some embodiments, the second catheter is an infusion catheter for infusing CSF into the subdural space, subarachnoid space, or ventricle. The subdural space is the anatomic space in the central nervous system beneath the inner surface of the dura mater and the outer arachnoid layer of the leptomeninges. The subdural space is commonly present only as a result of a disease, disorder, or injury, or when CSF is absent, thereby causing abnormal separation of the dura mater and the arachnoid mater. The subarachnoid space is the anatomic space in the central nervous system between the arachnoid mater and the pia mater. The ventricle is a cavity in the brain where the production and circulation of CSF takes place, and is referred to herein as the ventricle or brain ventricle.

In some embodiments, the CSF processing system includes one or more catheters. In some embodiments, the one or more catheters may be placed into the subdural space, subarachnoid space, or ventricle by placing one or more burr holes in the skull, and placing the one or more catheters into the subdural space, subarachnoid space, or ventricle in a corresponding burr hole.

In some embodiments, a dual lumen catheter is placed in any one of the subdural space, the subarachnoid space, or the brain ventricle in a single burr hole. In some embodiments, a first catheter may be placed in any one of the subdural space, the subarachnoid space, or the brain ventricle and a second catheter may be placed in any one of the subdural space, the subarachnoid space, or the brain ventricle. In yet another embodiment, a single burr hole may be placed in the skull, and both catheters may be placed into the skull through the single burr hole. In some embodiments, a first catheter is used for removal of CSF and a second catheter is used for infusion of CSF.

The intracranial pressure (ICP) is the pressure inside the skull, and under normal physiological conditions is in the range of 7 to 15 mmHg. ICP may be elevated as a result of a brain disease, disorder, or injury, which can be fatal if prolonged. On the other hand, decreased ICP can occur as a result of a leak of CSF, including as a result of lumbar punctures or other medical procedures that involve access to CSF, and can result in severe headaches, nausea, fatigue, or dizziness. Accordingly, it is important to monitor and maintain normal ICP levels during CSF processing and circulation through the CSF processing system. Thus, in some embodiments, the one or more catheters include a separate lumen design that allows for simultaneous drainage of CSF and monitoring of intracranial pressure. In some embodiments, the one or more catheters includes a pressure transducer that is unaffected by the flow rate, and provides for continuous monitoring of ICP. In some embodiments, the ICP is maintained at a physiologically normal range of 7, 8, 9, 10, 11, 12, 13, 14, or 15 mmHg, or within a range defined by any two of the aforementioned pressures. In some embodiments, the ICP is maintained at a decreased pressure of 1, 2, 3, 4, 5, or 6 mmHg, or within a range defined by any two of the aforementioned pressures.

Circulation Tubes

As used herein, the term “circulation tubes” refers to a tubing circuit through which CSF circulates. The tubing circuit is in fluid communication with the one or more catheters. In some embodiments, the circulation tubes are pre-primed with a balanced electrolyte solution and/or a balanced electrolyte solution can be added to the device, e.g., via one or more ports before, during or after circulation of the CSF. In some embodiments, the balanced electrolyte solution may comprise one or more compounds, such as acetate, gluconate, glucose (dextrose), albumin, lactate, creatinine, phosphorous, urea, sodium, potassium, chloride, calcium, or magnesium. In some embodiments, the balanced electrolyte solution comprises dextrose with one or more salts. In some embodiments, the balanced electrolyte solution comprises Normosol R™, Ringer's solution, Alsever's solution, Tyrode's solution, phosphate buffered saline, TRIS-buffered saline, PlasmaLyte-A™, Veen-D™, Polysal®, or Hank's balanced salt solution with or without dextrose or glucose.

In some embodiments, the pH of the balanced electrolyte solution is adjusted to a pH of 7.10, 7.15, 7.20, 7.25, 7.30, 7.35, 7.40, 7.45, 7.50, 7.55, 7.60, or 7.65 or within a range defined by any two of the aforementioned pH values. In some embodiments, the osmolarity of the balanced electrolyte solution is adjusted to an osmolarity of 250, 255, 260, 265, 270, 275, 280, 285, 290, 295, 300, 305, 310, 315, 320, 325, 330, 335, 340, or 345 mOsm/L or within a range defined by any two of the aforementioned values.

In some embodiments, the CSF circulation device maintains constant volume and constant ICP. In some embodiments, the tubing circuit is an extracorporeal tubing circuit, wherein CSF is circulated outside of the body of the subject. Accordingly, in some aspects is provided an extracorporeal CSF processing system, device, or method.

Pump

As used herein, the term “pump” refers to a device that controls fluid propulsion through the circulation tubes. In some embodiments, the pump is a roller-pump (peristaltic pump), a diaphragm pump, or a piston pump. The flow rate of CSF through the circulation tubes is controlled by the pump. In some embodiments, the flow rate is from 1 mL/min to 50 mL/min. Thus, in some embodiments, the flow rate is 1, 2, 3, 4, 5, 6, 7, 8, 9, 10, 15, 20, 25, 30, 35, 40, 45, or 50 mL/min, or within a range defined by any two of the aforementioned flow rates. In some embodiments, the pump comprises a positive and negative pressure feedback, which allows the pump to auto-regulate in response to negative pressure in the circulation tubing coming from the subject or positive pressure in the circulation tubing returning to the patient. In some embodiments, the pump further includes a cellular communications interface. In some embodiments, the pump works together in concert with the pressure sensors to maintain a flow rate that provides constant ICP, that decreases ICP (in cases of elevated ICP), or that increases ICP (in cases of low ICP).

Column

As used herein, the term “column” refers to a column for processing CSF. As used herein, the terms column and processing column are used interchangeably. The processing column is in fluid communication with the circulation tubing, wherein CSF passes through the circulation tubing and through the processing column. In some embodiments, the processing column is an affinity column, a capture column, a column containing binding agents, a size exclusion column, or multiple columns presented in tandem. Thus, in some embodiments, the CSF circulation device comprises one or more processing columns. In some embodiments, a membrane and/or filter are used either along with the column or in place of the column. In some embodiments, the column, membrane or filter comprises a support, such as beads or a resin or a polymer or a matrix. In some embodiments, the support is agarose, latex, acrylamide, magnetic bead, colored bead, or polymeric bead or nitrocellulose, polysulfone, or another polymer. In some embodiments, the support, column, membrane, or filter comprises a binding agent, such as a binding molecule, binding partner, or an affinity agent. In some embodiments, the column, membrane, filter, or support comprises a resin, wherein the resin includes Amberlite XAD7HP, Amberchrom CG300, an antibody, a binding fragment of an antibody, an extracellular matrix protein, such as hyaluronic acid, laminin, or fibronectin, a sorbent, an activated carbon, a ligand, an aptamer, or an immobilized biochemical compound or any combination thereof.

In some embodiments, the binding molecule or affinity agent present on the column, membrane, filter, or support comprises a lectin. In some embodiments, the lectin includes Galanthus nivalis agglutinin (GNA), Narcissus pseudonarcissus agglutinin (NPA), Concanavalin A or cyanovirin, Sambucus nigra lectin, ricin from Ricinus communis (RCA), Dolichos biflorus lectin, Ulex europaeus lectin, Vicia villosa lectin, Griffonia simplicifolia lectin, Solanium tuberosum lectin, Lycopersicon esculentum lectin, Datura stramonium lectin, Erythrina cristagalli lectin, Soybean lectin, Peanut agglutinin, Wheat germ agglutinin, phytohemagglutinin, griffithsin, or Jacalin lectin.

In some embodiments, the column, membrane, filter, or support comprises an affinity agent, for example, an antibody, such as a monoclonal antibody or a binding fragment thereof (e.g., a Fab fragment, ScFv, or a fragment having one or more CDR domains), that specifically bind to an antigen or epitope present on a molecular or pathogenic target. In some embodiments, the molecular or pathogenic target comprises a lipopolysaccharide, surface antigen (e.g., on the surface of microbes, such as bacteria or viruses or on the surface of cells, including neutrophils), proteins, debris, toxins, inflammatory mediators, and extracellular vesicles (e.g., exosomes). In some embodiments, debris includes cell debris (e.g., cell membranes), tissue debris (e.g., brain tissue or brain cells), or bone debris (e.g., bone fragments or bone cells). In some embodiments, the tissue or bone debris is present in CSF as a result of a penetrating cranial trauma, e.g., as a result of injury due to high velocity projectiles. Thus, in some embodiments, methods of removing cell membranes, brain tissue, brain cells or brain cell membranes, or bone tissue, bone cells, or bone cell membranes from a subject's CSF are provided, wherein a subject's CSF is introduced into a cerebrospinal fluid circulation device comprising: one or more catheters; at least one circulation tube in fluid communication with the one or more catheters; at least one column in fluid communication with the flow path; and at least one pump configured to circulate the cerebrospinal fluid through the flow path. Optionally, the method is employed in the aforementioned cerebrospinal fluid circulation device, wherein the device further comprises one or more filters and/or one or more columns configured to remove said cell membranes, brain tissue, brain cells or brain cell membranes, or bone tissue, bone cells, or bone cell membranes.

In some embodiments, the protein target comprises tau, β-amyloid, S100 β, neuron-specific enolase, glycoprotein A2B5, CD133, NQ01, synaptophysin, neuronal nuclei, MAB1569, polysialic acid-neural cell adhesion molecule (PSA-NCAM), or neurogenic differentiation 1 (NeuroD or Beta2), alpha synuclein, leucine-rich repeat kinase 2, superoxide dismutase enzymes or glycosylated or phosphorylated forms of these molecules, or other proteins associated with a brain disease, disorder, or injury.

In some embodiments, the inflammatory mediator to which the binding agent present on the column, membrane, filter, or support binds to is an endotoxin, lipopolysaccharide (LPS), a growth factor, an adhesion molecule, or a cytokine. In some embodiments, growth factors to which the binding agent present on the column, membrane, filter, or support binds to comprises, for example, DSL/jagged/notch families, EGF family, FGF family, Hedgehog family, IGF family, PDGF family, TGF-beta superfamily, VEGF family, wnt family, chondromodulin-1, CTGF/CCN2, Cyr 61/CCN1, EG-VEGF/PK 1, Fibulin 3, Fibulin 5/DANCE, HDGF, Hepassocin, HGF, LECT2, LEDGF, LRRN1/NCRR-1, LYAR, beta-NGF, Norrin, Nov/CCN3, oncomodulin, Osteocrin, PD-ECGF/Thymidine Phosphorylase, Progranulin/PGRN, S100A13, SLURP1, Thrombopoietin/TPO, or WISP-1/CCN4, or a combination thereof including any of their cofactors, splice variants, or binding proteins. In some embodiments, cytokines to which the binding agent present on the column, membrane, filter, or support binds to comprises, for example, adipocytokines, angiopoietin cytokines and angiopoietin-like protein families, chemokines and receptors, common beta-chain receptor family, common gamma-chain receptor family, interleukins, such as IL-2 and IL-8, IL-1 family, IL-6, IL-10, Interferon family, IP-10, IL-12 family, IL-17 family, TNF-superfamily, IL-13, IL-13R alpha 1, IL-13R alpha 2, IL-16, IL-32, IL-32 alpha, IL-32 beta, IL-32 gamma, or IL-34, or a combination thereof.

In some embodiments, the column, membrane, filter, or support comprises a lumen of a hollow fiber membrane, which forms an approach to accept and immobilize particulates from CSF. Thus, the column, membrane, filter, or support retains particulates bound by a binding agent while allowing CSF to pass through the lumen. Preferred binding agents for use with the CSF processing systems, devices, and methods include a binding agent such as, an antibody, such as a monoclonal antibody, or a binding fragment thereof (e.g., a Fab fragment, ScFv, or a fragment having a CDR domain), a lectin (e.g., Galanthus nivalis agglutinin (GNA), Narcissus pseudonarcissus agglutinin (NPA), Concanavalin A or cyanovirin, Sambucus nigra lectin, ricin from Ricinus communis (RCA), Dolichos billorus lectin, Ulex Europaeus lectin, Vicia villosa lectin, Griffonia simplicifolia lectin, Solanium tuberosum lectin, Lycopersicon esculentum lectin, Datura stramonium lectin, Erythrina cristagalli lectin, Soybean lectin, Peanut agglutinin, Wheat germ agglutinin, phytohemagglutinin, griffithsin, or Jacalin lectin), or an aptamer, such as a DNA aptamer that is specific for an antigenic site or epitope present on a tau, β-amyloid, S100 β, neuron-specific enolase, glycoprotein A2B5, CD133, NQ01, synaptophysin, neuronal nuclei, MAB1569, polysialic acid-neural cell adhesion molecule (PSA-NCAM), or neurogenic differentiation 1 (NeuroD or Beta2), alpha synuclein, leucine-rich repeat kinase 2, superoxide dismutase enzymes or glycosylated or phosphorylated forms of these molecules.

In some embodiments, the column, membrane, filter, or support used allow passage of viruses, prions, proteins, macromolecular debris, exosomes, toxins, or inflammatory mediators, or fragments thereof, but not most cellular components of contaminated CSF. In other embodiments, the membranes and filters used have pores that do not allow passage of viruses, prions, proteins, macromolecular debris, exosomes, toxins, or inflammatory mediators, or fragments thereof.

In particular embodiments, the column, membrane, filter, or support have pores that are 200-500 nm in diameter, more preferably, the pore size is 600 nm, which will allow passage of viruses, prions, proteins, macromolecular debris, exosomes, toxins, or inflammatory mediators, or fragments thereof, but not most cellular components of contaminated CSF, e.g., bacteria, protozoa, fungi, cells (red blood cells 10,000 nm diameter, lymphocytes 7,000-12,000 nm diameter, macrophages 10,000-18,000 nm diameter, thrombocytes 1000 nm). Optionally, by selecting a pore size that is smaller than the diameter of the cellular components of contaminated CSF, the membranes and filters exclude substantially all CSF contamination from passing through the pores and entering the extrachannel or extralumenal space of the column, membrane, filter, or support that contains the binding agent. In some embodiments, a pore size is selected that is smaller than only some particulate types.

In another embodiment, the column, membrane, filter, or support comprises a processing chamber having one or more binding agents disposed within the processing chamber, wherein said binding agents binds particles in the CSF. Preferred binding agents include a binding agent such as an antibody, such as a monoclonal antibody, or a binding fragment thereof (e.g., a Fab fragment, ScFv, or a fragment having one or more CDR domains), a lectin (e.g., Galanthus nivalis agglutinin (GNA), Narcissus pseudonarcissus agglutinin (NPA), Concanavalin A or cyanovirin, Sambucus nigra lectin, ricin from Ricinus communis (RCA), Dolichos billorus lectin, Ulex Europaeus lectin, Vicia villosa lectin, Griffonia simplicifolia lectin, Solanium tuberosum lectin, Lycopersicon esculentum lectin, Datura stramonium lectin, Erythrina cristagalli lectin, Soybean lectin, Peanut agglutinin, Wheat germ agglutinin, phytohemagglutinin, griffithsin, or Jacalin lectin), or an aptamer, such as a DNA aptamer that is specific for an antigenic site or epitope present on a tau, β-amyloid, S100 β, neuron-specific enolase, glycoprotein A2B5, CD133, NQ01, synaptophysin, neuronal nuclei, MAB1569, polysialic acid-neural cell adhesion molecule (PSA-NCAM), or neurogenic differentiation 1 (NeuroD or Beta2), alpha synuclein, leucine-rich repeat kinase 2, superoxide dismutase enzymes or glycosylated or phosphorylated forms of these molecules.

In some embodiments, the binding agent comprises proteins, for example, lectins, antibodies, antigens, extracellular matrix proteins, such as fibronectin, hyaluronic acid, or laminin. The technology to immobilize proteins in dialysis-like cartridges has been developed (Ambrus et al., Science 201(4358): 837-839, 1978; Ambrus et al., Ann Intern Med 106(4): 531-537, 1987; Kalghatgi et al. Res Commun Chem Pathol Pharmacol 27(3): 551-561, 1980, incorporated by reference in their entireties). An illustration of preparing proteins for immobilization to the hollow fibers for the method of the present invention is presented in U.S. Pat. Nos. 4,714,556, 4,787,974, and 5,528,057, incorporated by reference in their entireties.

For binding of binding agents, e.g., proteins, to the membrane, filter, or support, the polymers of the membrane, filter, or support are first activated, for example, made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41-3). Once the polymer has been activated, the proteins are attached directly or via a linker to form in either case an affinity matrix. Suitable linkers include, but are not limited to, avidin, streptavidin, biotin, protein A, and protein G. The proteins can also be directly bound to the polymer of the membrane using coupling agents such as bifunctional reagents, or can be indirectly bound. In one embodiment, the lectin, GNA, covalently coupled to agarose can be used to form an affinity matrix.

In some embodiments, a protein is attached to a substrate instead of, or in addition to, the membrane or filter. Suitable substrates include, but are not limited to, silica (e.g. glass beads, sand, diatomaceous earth) polysaccharides (e.g. dextran, cellulose, and agarose), proteins (e.g. gelatin), and plastics (e.g. polystyrenes, polysulfones, polyethersulfones, polyesters, polyurethanes, polyacrylates, nylons, carbon, and their activated and native amino and carboxyl derivatives). The protein can be bound to the substrates through standard chemical approaches, either directly, or through linkers such as C2 to C>20 linear and branched carbon chains, as well as the plastics, other proteins and polysaccharides listed above. For most synthetic purposes, C18 is the preferred upper limit but the chains can be added together for solubility reasons. Preferred linkers include: C2 to C18 dicarboxylates, diamines, dialdehydes, dihalides, and mixtures thereof (e.g. aminocarboxylates) in both native and activated form (e.g. disuccinimidyl suberimidate (DSS)). In some embodiments, one or more substrates can be used as linkers, alone or in combination with the substances listed as linkers. For example, dextran can be attached to sand, and additional linkers can then optionally be added to the dextran.

Filters

In some embodiments, the CSF processing system includes one or more filters for the removal of molecular targets and/or debris from CSF. In some embodiments, the filter is placed in the flow path of the circulation tube prior to CSF entry into the processing column. In some embodiments, the filter is placed in the flow path of the circulation tube after the CSF has entered the column. In some embodiments, the circulation device lacks a column but has a membrane or filter in the flow path of the circulation tube. In some embodiments, the one or more filters comprise one or more capsule filters or one or more disc filters, or a combination of capsule and disc filters. In some embodiments, the one or more filters have a pore size from 0.05 to 1.0 μm. In some embodiments, the one or more filters have pore sizes of 0.05, 0.1, 0.2, 0.3, 0.4, 0.5, 0.6, 0.7, 0.8, 0.9, or 1.0 μm or within a range defined by any two of the aforementioned pore sizes. In some embodiments, the filter has a 220 cm² surface area. In some embodiments, the filter is configured to remove molecular targets and cellular debris from CSF prior to CSF entering the processing column, and thus sterilizes CSF prior to entry to the column. In some embodiments, multiple filters are used in tandem. In some embodiments, the filter comprises a polymer material, such as a hydrophilic polyether sulfone membrane. In some embodiments, the filter is configured to process volumes ranging from 2 L to 50 L, and is configured to process CSF without changing the flow rate or ICP.

In some embodiments, the filter includes a plurality of filters, preferably, in tandem. For example, a filter can comprise a dual capsule filter, having two capsule filters in tandem. In some embodiments, the filter can be two or more filters in tandem, wherein each filter configured to remove and/or trap one or more different moieties. For example, a dual capsule filter may comprise two filters in tandem, whereby a first capsule filter is configured for removal of large molecules or particulate matter from CSF, and whereby a second capsule filter located downstream from the first capsule filter is configured to remove one or more smaller molecules, for example, particular molecules associated with inflammation and/or prions, tau, amyloid beta, alpha synuclein, leucine-rich repeat kinase 2, and/or superoxide dismutase enzymes. In some embodiments, a filter can comprise a size exclusion filter, an adsorption filter, or other filter comprising an affinity material bound thereto for capture of specific molecule of interest. In some embodiments, a filter can include various combinations of filters placed in various configurations combined into a single filtration unit for removal of particles and molecules from CSF. In some embodiments, the filter is configured to cleanse, purify, sterilize or remediate CSF, and/or for selective removal of one or more molecules of interest from CSF, such as prions, tau, amyloid beta, alpha synuclein, leucine-rich repeat kinase 2, and/or superoxide dismutase enzymes.

In some embodiments, the configuration of the one or more filters in tandem enables the ability to control or maintain flow of CSF through the CSF processing system at a constant rate or at variable rates. In some embodiments, the configuration of the one or more filters in tandem controls or reduces a clog or backup of CSF at the membrane or pores of the filter. In some embodiments, the configuration of the one or more filters in tandem enables filtration of larger biomolecules or particulate material. In some embodiments, configuration of the one or more filters in tandem allows for containment of affinity or binding material in a specified location while preventing the affinity or binding material from entering the circulating CSF.

Heat Exchanger

In some embodiments, the CSF processing system further comprises a heat exchanger for regulation of the temperature of CSF. In some embodiments, the heat exchanger is placed in the flow path of the circulation tube following or preceding the processing column. In some embodiments, the heat exchanger is configured to cool, maintain, or warm the temperature of the CSF. In some embodiments, the temperature of CSF is maintained at a range from 10 to 42° C. In some embodiments, the temperature of CSF is maintained at 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42° C. or within a range defined by any two of the aforementioned temperatures. Cooling of CSF provides for topical cooling of the brain and assists in reducing swelling and inflammation. Warming CSF provides for topical warming of the brain to influence blood flow and to alter metabolic activity of the brain or chemical activity of treatment.

In some embodiments, the CSF processing system further comprises an oxygenator. In some embodiments, CSF is oxygenated with an oxygen range from 21-100% oxygen, such as 21, 22, 23, 24, 25, 30, 35, 40, 45, 50, 55, 60, 65, 70, 75, 80, 85, 90, 95, or 100% or within a range defined by any two of the aforementioned percentages. In some embodiments, the oxygenator is combined with the heat exchanger as a single oxygenating-heat exchanging device. In some embodiments, the oxygenator accommodates a flow rate of up to 2 L/min. Although CSF does not contain hemoglobin as an oxygen carrying molecule, the oxygenator provides oxygen to CSF by providing dissolved oxygen, which provides oxygen to brain tissue thereby ameliorating ischemic events caused by injury to the brain, stroke, or other brain diseases, disorders, or injuries wherein oxygen delivery is compromised.

Infusion Port

In some embodiments, the CSF processing system comprises an infusion port. In some embodiments, the infusion port is configured to allow access to the CSF for purposes of sampling CSF or the introduction of an additive, compound, solution (e.g., an electrolyte solution, or medication. In some embodiments, CSF is removed from the CSF circulation system at the infusion port for the purpose of measuring the concentration of particles after the CSF has been processed. In some embodiments, the port provides an access point for administration of drugs for the treatment of a brain disease, disorder, or injury. In some embodiments, a treatment may include, for example, an antibody, an antibiotic, a chemotherapeutic, a steroid, or other drug for the treatment or amelioration of a specific brain disease, disorder, or injury. In some embodiments, the additive comprises exosomes, which comprise a medicament, a nucleic acid, or drug.

The type and quantity of medicament administered to a subject for the treatment of the brain disorder, disease, or injury is dependent on the type and severity of brain disorder, disease, or injury. Medicaments may include, for example, antibodies, antibiotics, chemotherapeutics, exosomes, which may be loaded with a therapeutic such as a drug or nucleic acid, or steroids. In some embodiments, the medicament comprises a drug delivery vehicle for delivery of an antibody, antibiotic, chemotherapeutic, or steroid to the brain. In some embodiments, the drug delivery vehicle comprises an exosome; a liposome, including, for example, a multivesicular liposome; a micelle; a polymeric nanoparticle; a dendrimer; or other drug delivery vehicle capable of delivering a drug payload. In some embodiments, the drug delivery vehicle comprises an exosome, wherein the exosome is repurposed for delivery of drugs. Exosomes are secreted nanoparticles that act as mediators in cellular communication and regulators of the cellular niche. Exosomes secretion and characteristics are altered in many diseases, such as cancer. Accordingly, in some embodiments, exosomes are isolated and removed from contaminated CSF and repurposed by loading exosomes with an appropriate drug for delivery to the subject.

Administration of a medicament or a drug delivery vehicle containing the medicament using the CSF processing system described herein bypasses the BBB and thereby provides efficient delivery to the target tissue.

### Methods of Processing Cerebrospinal Fluid

Provided herein are methods for processing cerebrospinal fluid using a CSF processing system as described herein. In some embodiments, one or more catheters are placed into the subdural space, subarachnoid space, or ventricle of a subject, and CSF flows through the CSF processing system. In some embodiments, CSF is processed by passage through one or more processing columns, membranes, filters, or supports, wherein the CSF is processed and/or cleansed or clarified. In some embodiments, the processed CSF is returned to the subject. In some embodiments, processed CSF has reduced levels of particulate contamination, which includes, for example, viruses, bacteria, fungi, protozoa, parasites, prions, proteins, cells, debris, exosomes, toxins, or inflammatory mediators, or fragments or combinations thereof. In some embodiments, CSF is processed by increasing or decreasing the temperature, by increasing the oxygen levels, by removal of particulate contamination, or by administration of a drug, or combinations thereof. The type of processing will be dependent on the specific brain disease, disorder, or injury, and will depend of the clarity or purity of CSF, or the degree of contamination of CSF.

### Methods of Treating Pathological Conditions

Provided herein are methods for treating pathological conditions by circulating CSF in a CSF processing system described herein. In some embodiments, the system is configured to allow a subject's CSF to be circulated outside of the body, made available for treatment, and returned to the body in real time. While outside of the body, CSF can be processed in various ways depending on the pathology on seeks to ameliorate. Various disease conditions, disorders, and injuries can affect the CSF, resulting in an increased contamination of CSF. For example, brain diseases, disorders, or injuries may include, but are not limited to cerebral vasospasm, Alzheimer's disease, Huntington's disease, Guillain Barre Syndrome, Parkinson's disease, amyotrophic lateral sclerosis, multiple sclerosis, spinal cord injury, traumatic brain injury, chronic traumatic encephalopathy, stroke, cancer affecting the brain or spinal cord, prion disease, encephalitis, meningitis, or enzymatic or metabolic imbalances that result in a secondary brain disorder, or other diseases that result in contamination of CSF.

In some embodiments, the brain disorder or disease is a brain cancer or brain tumor. Examples of brain cancers and brain tumors include, anaplastic astrocytomas, glioblastoma, meningioma, mesenchymal tumors, astrocytoma, pituitary tumors, schwannomas, CNS lymphoma, oligodendrogliomas, ependymomas, low-grade astrocytomas, medulloblastomas, astrocytic tumors, pilocytic astrocytomas, diffuse astrocytomas, pleomorphic xanthoastrocytomas, subependymal giant cell astrocytomas, oligodendroglial tumors, anaplastic oligodendrogliomas, oligoastrocytomas, anaplastic oligoastrocytomas, myxopapillary ependymomas, subependymomas, ependymomas, anaplastic ependymomas, astroblastomas, chordoid gliomas of the third ventricle, gliomatosis cerebris, gangliocytomas, desmoplastic infantile astrocytomas, desmoplastic infantile gangliogliomas, dysembryoplastic neuroepithelial tumors, central neurocytomas, cerebellar liponeurocytomas, paragangliomas, ependymoblastomas, medulloblastomas, supratentorial primitive neuroectodermal tumors, choroids plexus papilloma, pineocytomas, pineoblastomas, pineal parenchymal tumors of intermediate differentiation, hemangiopericytomas, melanocytic lesions, germ cell tumors, tumors of the sellar region, craniopharyngioma, capillary hemangioblastoma, and primary CNS lymphoma. Accordingly, provided herein is a method of treating or inhibiting a brain cancer or brain tumor. In some embodiments, the method comprises introducing fluid from a subject suffering from a brain cancer or brain tumor into a CSF circulation device as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device may comprise cellular debris, cytokines, signaling molecules, or toxins. In some embodiments, the CSF is returned to the subject having less particulate matter than it had prior to introduction into the device. In some embodiments, the CSF undergoes further processing by introducing a medicament for treatment, such as a chemotherapeutic or steroid, or a drug delivery vehicle comprising a chemotherapeutic or steroid. In some embodiments, the CSF is processed by increasing or decreasing the temperature of the CSF.

In some embodiments a method of treating or inhibiting a bacterial or viral infection of the central nervous system or brain is provided. In some embodiments, the method comprises introducing fluid from a subject suffering from a bacterial or viral infection of the CSF or brain into a CSF circulation device as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device comprises bacteria, viruses, or fragments thereof. In some embodiments, the CSF is returned to the subject having less particulate matter than it had prior to introduction into the device. In some embodiments, the CSF undergoes further processing by introducing a medicament for treatment or amelioration of the bacterial or viral infection. In some embodiments, the CSF is processed by increasing or decreasing the temperature of the CSF.

Meningitis refers to an inflammatory process of the leptomeninges and CSF within the subarachnoid space. Meningitis is usually caused by an infection, but chemical meningitis may also occur in response to a nonbacterial irritant introduced into the subarachnoid space. Infectious meningitis is classified broadly as acute pyogenic, aseptic, and chronic. Chronic meningitis comprises, for example, tuberculous, spirochetal, or cryptococcal. In some embodiments, a method of treating or ameliorating meningitis is provided. In some embodiments, the method comprises introducing fluid from a subject suffering from meningitis into a CSF circulation device, as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device comprises bacteria, viruses, or fragments thereof. In some embodiments, the CSF is returned to the subject having less particulate matter than it had prior to introduction into the device. In some embodiments, the CSF undergoes further processing by introducing a medicament for treatment or amelioration of the bacterial or viral infection. In some embodiments, the CSF is processed by increasing or decreasing the temperature of the CSF.

Acute pyogenic meningitis is caused by bacterial infections, which can include, for example, Escherichia coli, Streptococcus pneumoniae, Listeria monocytogenes, Neisseria meningitidis, Haemophilus influenzae, and other bacterial infections common to the central nervous system. In subjects having infectious bacterial meningitis, the CSF is cloudy or purulent, and the subject experiences increased ICP. In some embodiments, infected CSF may have 1000, 2000, 3000, 4000, 5000, 6000, 7000, 8000, 9000, 10000, 15000, 20000, 25000, 30000, 40000, 50000, 60000, 70000, 80000, or 90000 microorganisms/mm³, or a value within a range defined by any two of the aforementioned values. In some embodiments, infected CSF has a raised protein level, a reduced glucose content, or a combination thereof. In some embodiments a method of treating acute pyogenic meningitis is provided. In some embodiments, the method includes introducing fluid from a subject suffering from acute pyogenic meningitis into a CSF circulation device as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device comprises bacteria, viruses, or fragments thereof. In some embodiments, the CSF is returned to the subject having less particulate matter than it had prior to introduction into the device. In some embodiments, the CSF undergoes further processing by introducing a medicament for treatment or amelioration of the bacterial or viral infection. In some embodiments, the CSF is processed by increasing or decreasing the temperature of the CSF.

Acute aseptic meningitis is caused by viral infections, which can include, for example, enterovirus, echovirus, coxsackievirus, and other viral infections common to the central nervous system. The CSF in viral meningitis may be marked with an increase in cell count (pleocytosis), such as an increase in white blood cell count, an increase in protein levels, or a combination thereof. In some embodiments, a method of treating acute aseptic meningitis is provided. In some embodiments, the method comprises introducing fluid from a subject suffering from acute aseptic meningitis into a CSF circulation device as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device comprises bacteria, viruses, or fragments thereof. In some embodiments, the CSF is returned to the subject having less particulate matter than it had prior to introduction into the device. In some embodiments, the CSF undergoes further processing by introducing a medicament for treatment or amelioration of the bacterial or viral infection. In some embodiments, the CSF is processed by increasing or decreasing the temperature of the CSF.

Encephalitis refers to an acute inflammation of the brain that may be the direct effect of an acute infection, or as a one of the sequelae of a latent infection. Encephalitis with meningitis is meningoencephalitis. Bacterial meningoencephalitis included tuberculosis. The CSF of tuberculosis subjects has moderate CSF pleocytosis, which includes mononuclear cells or a mixture of mononuclear cells and polymorphonuclear cells. The CSF also has elevated protein levels, and moderately reduced glucose levels. Viral encephalitis is inflammation of the brain almost invariably associated with a meningeal inflammation, with moderate CSF pleocytosis, elevated protein levels, and elevated pressure. In some embodiments, a method of treating encephalitis is provided. In some embodiments, the method comprises introducing fluid from a subject suffering from encephalitis into a CSF circulation device as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device comprises bacteria, viruses, or fragments thereof. In some embodiments, the CSF is returned to the subject having less particulate matter than it had prior to introduction into the device. In some embodiments, the CSF undergoes further processing by introducing a medicament for treatment or amelioration of the bacterial or viral infection. In some embodiments, the CSF is processed by increasing or decreasing the temperature of the CSF.

Another disorder associated with changes in CSF includes traumatic brain injury (TBI). In the United States, at least 1.7 million people sustain a traumatic brain injury each year. Injuries to the brain result in a variety of outcomes including 52,000 deaths, 275,000 hospitalizations, and 1,365,000 individuals treated and released from an emergency department. In children from 0 to 14 years of age, brain injuries each year result in approximately 2,685 deaths, 37,000 hospitalizations, and 435,000 emergency department visits. All brain injuries are unique and the type of injury the brain receives may be limited to one functional area of the brain, or various areas, or all areas of the brain. CSF of injured brains can include elevated protein levels (including, for example, elevated tau), pleocytosis, cellular debris, decreased oxygenation, or increased ICP as a result of the injury. In addition, depending on the type of injury, the brain may experience swelling. Chronic traumatic encephalopathy (CTE) is a progressive neurodegenerative disease associated with prior exposure to repetitive head impacts, primarily through contact sports.

In some embodiments, a method of treating or ameliorating traumatic brain disorders (including traumatic brain injuries) is provided. In some embodiments, the method comprises introducing CSF from a subject suffering from traumatic brain disorders into a CSF circulation device as described herein. In some embodiments, the traumatic brain disorder includes CTE. In some embodiments, the CSF is circulated through the CSF circulation device. In some embodiments, the CSF is processed by removing particulate matter, such as proteins (such as tau, amyloid beta, prions, or fragments or combinations thereof) or cellular debris from the CSF (e.g., by binding the particulate matter on a column, membrane, filter, or support that is in the flow path of the device). In some embodiments, the CSF is returned to the subject having less particulate matter than it had prior to introduction into the device. In some embodiments, the CSF is further processed by introducing a medicament for treatment or amelioration of traumatic brain disorders. In some embodiments, the CSF is processed by increasing or decreasing the temperature of the CSF. In some embodiments, the CSF is processed by increasing the oxygenation levels or by decreasing the ICP.

Brain abscesses may also arise as a result of organismal infection, sometimes as a result of a local infection or from hematogenous spread. Subjects with brain abscesses have CSF that is under increased pressure, elevated white blood cell count, and elevated protein levels. Abscesses generally require surgical treatment and administration of antibiotics. In some embodiments, a method of treating abscesses is provided. In some embodiments, the method includes introducing fluid from a subject suffering from abscesses into a CSF circulation device as described herein. In some embodiments, the CSF is circulated through the CSF circulation device. In some embodiments, the CSF is processed, cleansed, or clarified by removing particulate matter (e.g., by binding the particulate matter on a column, membrane, filter, or support that is in the flow path of the device). The particulate matter that can be removed by the device comprises bacteria, viruses, or fragments thereof. In some embodiments, the CSF is returned to the subject having less particulate matter than it had prior to introduction into the device. In some embodiments, the CSF undergoes further processing by introducing a medicament, such as an antibody, for treatment or amelioration of the organismal infection. In some embodiments, the CSF is processed by increasing or decreasing the temperature of the CSF.

Other diseases of the brain that affect the CSF include diseases caused by fungi, parasites, protozoa, or prions. In each of these cases, the target pathology may be present in the CSF, and furthermore, may result in elevated levels of protein, cells, toxins, or inflammatory mediators, and may result in increased ICP.

Accordingly, provided herein is a method of processing CSF in a subject in need, wherein the method includes removing the CSF from the subject using an CSF circulation system as described herein, processing the CSF, and returning the processed CSF to the subject. In some embodiments, the removed CSF is passed through one or more columns, membranes, or filters (e.g., an affinity column) so as to process, cleanse, or clarify the CSF. In some embodiments, the one or more columns, membranes, or filters remove particulate matter, which may include one or more of viruses, bacteria, fungi, parasites, protozoa, prions, proteins, cells, toxins, or inflammatory mediators, or fragments thereof. In some embodiments, the CSF circulation system includes a warmer/cooler for processing the CSF, wherein the CSF is warmed or cooled. In some embodiments, the CSF is cooled in order to reduce swelling. In some embodiments, the CSF circulation system includes an oxygenator for increasing the oxygenation level of the CSF. In some embodiments, the CSF circulation system is configured to decrease the ICP of the subject. In some embodiments, the CSF is reinfused with a medicament.

In some embodiments, the CSF circulation system comprises a port for removal of CSF from the system and/or for the addition of material to the CSF. In some embodiments, CSF is removed from the system for further processing. In some embodiments, one or more medications are administered to the subject by adding the one or more medications into the port. Accordingly, the CSF circulation system provides for a mechanism for drug delivery. Drugs for delivery to the CSF may include, for example, antibiotics, antibodies, chemotherapeutics, or steroids. In some embodiments, the additive comprises exosomes, which comprise a medicament, a nucleic acid, or drug. In this way, drugs may be delivered to the brain through the CSF, allowing an access port for bypassing the blood brain barrier.

In some embodiments, a method of treating or ameliorating a brain disorder or a brain disease is provided, wherein CSF of a subject or patient is processed using a CSF circulation system described herein. In some embodiments, the disease or disorder comprises, for example, viral meningitis, bacterial meningitis, viral encephalopathy, bacterial encephalopathy, traumatic brain injury, abscesses, an infections caused by fungi, protozoa, parasites, prions, bacteria, and/or viruses, and combinations thereof, or other brain disorders that result in changes to the CSF, including increased particulate matter or increased ICP. In some embodiments, the CSF is processed, cleansed, or clarified by removing the particulate matter from the CSF. In some embodiments, the particulate matter includes viruses, bacteria, fungi, protozoa, parasites, prions, proteins, exosomes, cells, toxins, inflammatory cytokines, or combinations thereof. In some embodiments, the CSF is processed by cooling or warming. In some embodiments, the CSF is processed by increasing oxygen levels of the CSF. In some embodiments, the method of treating or ameliorating a brain disorder or a brain disease further comprises returning the processed CSF to the subject. In some embodiments, the CSF is returned with the addition of a medicament.

The invention is generally disclosed herein using affirmative language to describe the numerous embodiments. The invention also includes embodiments in which subject matter is excluded, in full or in part, such as substances or materials, method steps and conditions, protocols, or procedures. Thus, even though the invention is generally not expressed herein in terms of what the invention does not include aspects that are not expressly excluded in the invention are nevertheless disclosed herein.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Cerebrospinal Fluid Circulation System

The following example provides one embodiment of a CSF circulation system as described herein.

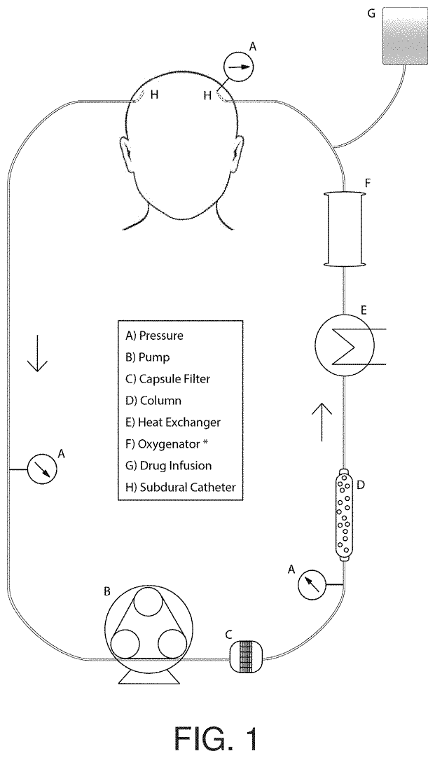

FIG. 1 illustrates one embodiments of a CSF circulation system. In this embodiment, subdural drainage catheters (H) are used as cannula for access to CSF. ICP is monitored with pressure transducers within the catheters. The pressure is monitored at various locations throughout the system by pressure sensors (A), which are located upstream and downstream of the filter (C), and downstream and upstream of the column (D). Pressure sensors detect negative and positive pressure, and are used to maintain, decrease, or increase pressure, depending on the circumstance and requirements for the specified indication.

The system includes tubing or a flow path through which CSF circulates and is processed. The system is primed with a balanced electrolyte/dextrose solution. Pumps (B), such as peristaltic pumps propel CSF through the circuit, and control the flow rate. One or more filters (C) can be used throughout the system to sterilize CSF. For example, the filter (C) can be a 0.2-micron capsule filter or a disc filter located prior to the processing column (D). The processing column is a column as described herein.

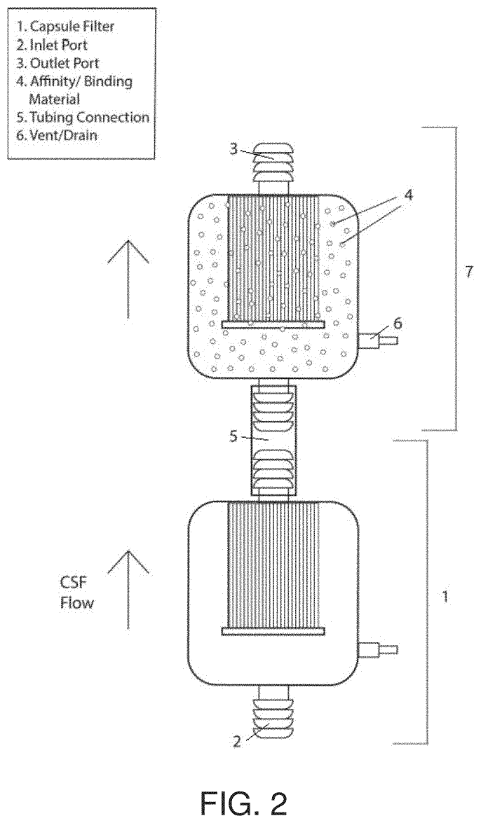

In some embodiments, the filter can be a dual capsule filter, such as a dual capsule filter shown in FIG. 2. In the embodiment of FIG. 2, CSF flows through a dual capsule filter unit into an inlet port (2) to a first capsule filter. In some embodiments, a first capsule filter (1) removes any large molecules or particulate matter from CSF. In some embodiments, CSF flows through the first capsule filter (1) to a second capsule filter (7) having affinity/binding material (4) located therein. In some embodiments, affinity or binding material (4) in the second capsule filter (7) captures or retains molecules of interest, and CSF now lacking particulate or large molecules captured in the first capsule filter and lacking molecules of interest captured in the second capsule filter exits through the outlet port (3).

Also included in the circulation system is a heat exchanger (E) and an oxygenator (F). The system also includes a port (G) for infusion of medication or for the removal of CSF for testing or for further assessment.

One of skill in the art will recognize that the system can include various combinations of elements or components as described herein. For example, one component can be placed in a different order or location as provided in the embodiment of FIG. 1. In addition, one component may be included in multiple locations, for example, the use of multiple filters, or multiple columns used in tandem.

Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference. The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the present invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Furthermore, although the foregoing has been described in some detail by way of illustrations and examples for purposes of clarity and understanding, it will be understood by those of skill in the art that numerous and various modifications can be made without departing from the spirit of the present disclosure.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

1. A cerebrospinal fluid circulation device comprising:
one or more catheters;
at least one circulation tube in fluid communication with the one or more catheters;
at least one column in fluid communication with the flow path; and
at least one pump configured to circulate the cerebrospinal fluid through the flow path.

2-62. (canceled)
